(19) United States
(12) Patent Application Publication
(10) Pub. No.: US 2006/0292610 A1
(43) Pub. Date: Dec. 28, 2006

(54) Prognostic meta signatures and uses thereof

(75) Inventors: Ronglai Shen, Ann Arbor, MI (US); Debashis Ghosh, Ann Arbor, MI (US); Arul Chinnaiyan, Plymouth, MI (US)

Correspondence Address: Medlen & Carroll, LLP, Suite 350, 101 Howard Street, San Francisco, CA, 94105, US

(73) Assignee: Regents of the University of Michigan, Ann Arbor, MI (US)

(21) Appl. No.: 11/447,836

(22) Filed: Jun. 6, 2006

Related U.S. Application Data
(60) Provisional application No. 60/687,764, filed on Jun. 6, 2005.

(51) Int. Cl. C12Q 1/68; G06F 19/00

(52) U.S. Cl. 435/6; 702/20

(57) ABSTRACT

The present invention relates the novel statistical analyses of gene expression data. In particular, the present invention provides prognostic meta signatures (e.g. representing cancer samples) that identify genes with altered expression across several studies.

The present application claims priority to U.S. Provisional Application Ser. No. 60/687,764, filed Jun. 6, 2005, herein incorporated by reference in its entirety.

This work was supported in part by grants from the National Institutes of Health and National Science Foundation grant Nos. R01 GM72007, R01 CA97063, and P50 CA69568. The government may have certain rights in the invention.

### FIELD OF THE INVENTION

The present invention relates to the statistical analyses of gene expression data. In particular, the present invention provides prognostic meta signatures (e.g. representing cancer samples) that identify genes with altered expression across several studies.

### BACKGROUND OF THE INVENTION

DNA microarray analysis has been shown to be a powerful tool in various aspects of research, including cancer (Greer et al., Ann N.Y. Acad Sci 2004, 1020:49-66). Expression profile analysis provides information on genes whose expression is altered in a disease state. Such genes are valuable targets for research and drug discovery.

With the increasing availability of published microarray data sets, there is a tremendous need to develop approaches for validating and integrating results across multiple studies. A major concern in the meta-analysis of DNA microarrays is the lack of a single standard experimental platform for data generation. Expression profiling data based on different technologies can vary significantly in measurement scale and variation structure. It poses a great challenge to compare and integrate results across independent microarray studies. The large variability brought in by microarray datasets using different platforms is expected to affect the sensitivity and specificity of summary statistics constructed in various ways across studies. Given the inherent differences of the microarray techniques, heterogeneity of the sample populations, and low comparability of the independently generated data sets, meta-analysis of microarrays remains a difficult task.

What is needed in the art are new methods for the analysis and integration of multiple expression profile data sets.

### SUMMARY OF THE INVENTION

The present invention relates the novel statistical analyses of gene expression data. In particular, the present invention provides prognostic meta signatures (e.g. representing cancer samples) that identify genes with altered expression across several studies.

Accordingly, in some embodiments, the present invention provides methods of analyzing multiple expression profile studies. In some preferred embodiments, the expression profiles compare disease to non-disease states. In some embodiments, the present invention provides prognostic meta signatures representing combined expression data sets and methods of generating such signatures. The prognostic meta signatures of the present invention find use in characterizing, diagnosing, and providing prognostic information for a variety of diseases (e.g., cancer). The prognostic meta signatures of the present further find use in research applications (e.g., drug screening).

For example, in some embodiments, the present invention provides a method, comprising: providing a plurality of microarray data sets, wherein each data set represents microarray profiling of a distinct sample, and wherein the sample is representative of a disease state; performing a two stage Bayesian mixture modeling calculation on the data sets to generate probability of expression (poe) matrices for each data set; combining the poe matrices to generate a combined data matrix; and generating a prognostic meta signature for the disease state based on the combined data matrix. In some embodiments, the disease is cancer (e.g., breast cancer). In some embodiments, the microarray data sets comprise gene expression data. In some embodiments, the prognostic signature comprises expression data for at least 20, preferably at least 50, even more preferably at least 100, and still more preferably at least 500 genes. In some embodiments, the prognostic meta signature is indicative of increased or decreased probability of relapse free survival of cancer.

The present invention further provides a prognostic meta signature comprising normalized gene expression data from at least two (e.g., at least 3) independent gene expression profiling studies, wherein the gene expression data comprises data from microarray profiling of a distinct sample, and wherein the sample is representative of a disease state. In some embodiments, the prognostic meta signature is indicative of increased or decreased probability of relapse free survival of cancer. In some embodiments, the prognostic signature comprises expression data for at least 20, preferably at least 50, even more preferably at least 100, and still more preferably at least 500 genes. In some embodiments, the prognostic meta signature combined probability of expression matrices from the at least 2 independent gene expression profiling studies. In some embodiments, the probability of expression matrices are generated using two stage Bayesian mixture modeling calculation on normalized data sets from the at least 2 independent gene expression profiling studies. In some embodiments, the disease is cancer (e.g., breast cancer).

In yet other embodiments, the present invention provides a method of screening compounds, comprising: providing a cell; and one or more test compounds; and contacting the cell with the test compound; generating a gene expression profile of the cell in the presence and absence of the test compound; and comparing the gene expression profile to a prognostic meta signature generated by the method described herein. In some embodiments, the cell is a cancer cell. In some embodiments, the prognostic meta signature represent gene expression profiles of cancer cells. In some embodiments, the cell is in an animal (e.g., a human or a non-human mammal).

### BRIEF DESCRIPTION OF THE DRAWINGS

FIG. 1 shows a schematic of meta-analysis of microarray data using a two-stage mixture model approach.

FIG. 2 shows that the 90-gene meta-signature displayed greater performance than nodal status in predicting relapse-free survival in breast cancer, and it further predicts survival outcome in nodal status sub-cohorts. (A) Lymph node status correlates with survival outcome (P=0.0004). (B) The meta-signature correlates with survival outcome (P=2×10−10). (C) The meta-signature differentiates risk groups in nodal negative patients (P=2.6×10−5). (D) The meta-signature predicts risk groups in nodal positive patients (P=7.0×10−5).

FIG. 3 shows that the 90-gene meta-signature achieves similar or better performance than the individually optimized signatures. A and E compare the Kaplan-Meier curves stratified by high versus low risk group predicted by the study-specific signature and by the meta signature respectively in the Sorlie study cohort; B and F show similar comparison in the van't Veer study cohort; C and G show similar comparison in the Sotiriou study cohort; and D and H show comparison in the Huang study cohort.

FIG. 4 shows a comparison of model performances based on data integrated by poe transformation (A and C) and global standardization (B and D).

FIG. 5 shows the top seven over-represented functional classes in the meta-signature.

### DEFINITIONS

To facilitate an understanding of the present invention, a number of terms and phrases are defined below:

As used herein, the term “prognostic meta signature” refers to a set of data (e.g., gene expression data) that encompasses multiple sets of data derived from independent studies that is normalized to a common scale. In some preferred embodiments, the common scale is a probability of expression (poe) scale. A prognostic meta signature normalizes multiple studies that, due to heterogeneity of patient cohorts and sensitivity/specificity of measurement technology, may not identify the same variables (e.g., differentially expressed genes).

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

As used herein, the term “subject” refers to any animal (e.g., a mammal), including, but not limited to, humans, non-human primates, rodents, and the like, which is to be the recipient of a particular treatment or research application. Typically, the terms “subject” and “patient” are used interchangeably herein in reference to a human subject.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis (e.g., a CT scan showing a mass or increased PSA level) but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental expose, previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes (e.g., by utilizing the prognostic meta signatures described herein).

As used herein, the term “characterizing tissue in a subject” refers to the identification of one or more properties of a cancer tissue sample (e.g., including but not limited to, the presence of cancerous tissue, the presence of pre-cancerous tissue that is likely to become cancerous, and the presence of cancerous tissue that is likely to metastasize). In some embodiments, tissues are characterized by the identification of the expression of one or more cancer marker genes (e.g., by utilizing the prognostic meta signatures described herein).

As used herein, the term “cancer marker genes” refers to a gene whose expression level, alone or in combination with other genes, is correlated with cancer or prognosis of cancer. The correlation may relate to either an increased or decreased expression of the gene. For example, the expression of the gene may be indicative of cancer, or lack of expression of the gene may be correlated with poor prognosis in a cancer patient.

As used herein, the term “a reagent that specifically detects expression levels” refers to reagents used to detect the expression of one or more genes (e.g., including but not limited to, the cancer markers of the present invention). Examples of suitable reagents include but are not limited to, nucleic acid probes capable of specifically hybridizing to the gene of interest, PCR primers capable of specifically amplifying the gene of interest, and antibodies capable of specifically binding to proteins expressed by the gene of interest. Other non-limiting examples can be found in the description and examples below.

As used herein, the term “instructions for using said kit for detecting cancer in said subject” includes instructions for using the reagents contained in the kit for the detection and characterization of cancer in a sample from a subject. In some embodiments, the instructions further comprise the statement of intended use required by the U.S. Food and Drug Administration (FDA) in labeling in vitro diagnostic products.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

As used herein, the term “providing a prognosis” refers to providing information regarding the impact of the presence of cancer (e.g., as determined by the diagnostic methods of the present invention) on a subject's future health (e.g., expected morbidity or mortality, the likelihood of getting cancer, and the risk of metastasis).

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention.

As used herein, the term “initial diagnosis” refers to results of initial cancer diagnosis (e.g. the presence or absence of cancerous cells). An initial diagnosis does not include information about the stage of the cancer of the risk of prostate specific antigen failure.

As used herein, the term “biopsy tissue” refers to a sample of tissue (e.g., prostate tissue) that is removed from a subject for the purpose of determining if the sample contains cancerous tissue. In some embodiment, biopsy tissue is obtained because a subject is suspected of having cancer. The biopsy tissue is then examined (e.g., by microscopy) for the presence or absence of cancer.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely-homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

As used herein the term “portion” when in reference to a protein (as in “a portion of a given protein”) refers to fragments of that protein. The fragments may range in size from four amino acid residues to the entire amino acid sequence minus one amino acid.

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally occurring gene.

As used herein, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### DETAILED DESCRIPTION OF THE INVENTION

Expression profiling data based on different technologies can vary significantly in measurement scale and variation structure. It poses a great challenge to compare and integrate results across independent microarray studies. In a recent study of diffuse large B cell lymphoma (DLBCL), Wright et al. (Wright et al., Proc Natl Acad Sci 2003, 100:9991-6) sought to bridge two different microarray platforms by validating findings from a cDNA lymphochip microarray using an independent dataset generated using Affymetrix oligonucleotide arrays; Although the idea of training and testing classifiers is frequently used for discriminant analysis, this application to distinct expression array platforms is less common. More systematic approaches have been proposed for integration of findings from multiple studies using different array technologies. Rhodes et al. (Rhodes et al., Cancer Research 2002, 62:4427-33) have proposed methods to summarize significance levels of a gene in discriminating cancer versus normal samples across multiple gene profiling studies. By ranking the q-values (Storey, J R Stat Soc B 2002, 64:479-98) from sets of combinations, a cohort of genes from the four studies was identified to be abnormally expressed in prostate cancer. Choi et al. (Choi et al., Bioinformatics 2003, 19:i84-i90) suggested combining effect size using a hierarchical model, where the estimated effect size in individual studies follows a normal distribution with mean zero and between study variance τ2. The effect size was defined to be the difference between the tumor and normal sample means divided by pooled standard deviation. From a Bayesian perspective, other studies used data from one study to generate a prior distribution of the differences in logarithm of gene expression between diseased and normal groups, and subsequent microarray studies updated the parameter values of the prior. Assuming a normal error distribution, the differences were then combined to form a posterior mean. Although phrased using different model frameworks, these methods are similar in the spirit of combining the standardized differences between two sample means across multiple studies. It has been shown, however, that the overlap between significant gene detection on different array platforms is only moderate due to low comparability of independent data sets (Mah et al., Physiol Genomics 2004, 16:361-70). The large variability brought in by microarray datasets using different platforms is expected to affect the sensitivity and specificity of summary statistics constructed in various ways across studies. Given the inherent differences of the microarray techniques, heterogeneity of the sample populations, and low comparability of the independently generated data sets, meta-analysis of microarrays remains a difficult task.

In some embodiments of the present invention a Bayesian mixture model is used to estimate the probability of over-, under- or baseline expression for gene sample combinations given the observed expression measurements. With data-driven estimation of these quantities, one can translate the raw expression measurement into a probability of differential expression. As a result, poe (i.e., probability of expression) was introduced as a new scale and used in the context of molecular classification. The platform-free property of this scale allowed experiments conducted during the course of development of the present invention to incorporate poe in a framework to meta-analyze microarray data. Several desirable features of using poe as a new expression scale include the following: 1. poe provides a scaleless measure and thereby facilitates data integration across microarray platforms; 2. poe is a model-based transformation with direct biological implications in the context of gene expression data, as it is estimated based on a method that adopts an underlying mixture distribution that accommodates over-, under-, and unchanged expression categories.; 3. poe unmasks differential expression patterns in microarray data by offsetting the influence of extreme expression values (Scharpf et al., BioTechniques 2003, 34:S22-S29); 4. Data integration based on poe allows merging of samples on the unified scale rather than using gene-specific summaries.

In recent publications of breast cancer microarray studies, several groups have explored the hypothesis that the capacity to metastasize is intrinsic to the tumor and therefore can be revealed by gene expression pattern. Four independent studies have correlated gene expression profiles generated from distinct DNA microarray platforms to breast cancer prognosis (Sorlie et al., Proc Natl Acad Sci 2001, 98:10869-74; van't Veer et al., Nature 2002, 415:530-6; Sotiriou et al., Proc Natl Acad Sci 2003, 100:10393-8; Huang et al., Lancet 2003, 361:1590-6). Among the four, Sorlie et al. (supra) and Sotiriou et al. (supra), both cDNA microarray studies, applied unsupervised clustering and identified several breast cancer subtypes characterized by differential expression of a cohort of genes. Further, they correlated the tumor subtypes derived from the expression profile with survival outcome and in both cases found that, as expected, the ERBB2+ subtype correlated with shorter survival times. On the other hand, van't Veer et al. (supra), an inkjet oligonucleotide array study, and Huang et al. (supra), an Affymetrix GeneChip study, have built classification models based on gene expression profiles to predict 5-year or 3-year recurrence status. In all four studies, however, the authors explored a common hypothesis that molecular profiles were able to provide a more accurate prediction of patient survival compared with clinical/pathological parameters. These studies therefore provided an excellent basis for developing a meta-analysis of microarrays with regard to disease prognosis.

Experiments conducted during the course of development of the present invention demonstrated the use of a two-stage meta-analysis of microarrays based on poe. The method was applied to the aforementioned breast cancer DNA microarray data sets. With the strength of the poe transformation and data integration, an inter-study validated meta-signature that predicts relapse free survival in breast cancer patients with improved statistical power and reliability was developed.

Factors relevant to integrating microarray studies include use of different gene expression measurement scales, varying analytical power and reliability of the results for individual studies. To account for these issues, a two-stage mixture modeling strategy was utilized, the strength of which was built on the mixture model based transformation and the subsequent data integration on the poe scale. In particular, poe provides a unified platform-free scale, and simultaneously enhances the intrinsic contrast in the expression data. Furthermore, combining sample pools on the poe scale mitigates the influence of potential artifacts from a single study. The benefit of such data integration is reflected on two counts. One, integrated sample cohorts improve the reliability of the findings by guarding against false positive results from a single study. Two, it increases the statistical power to detect small consistent effects that can be otherwise masked by inadequacy of the sample size of an individual data set. By implementing this modeling approach, experiments conducted during the course of development of the present invention combined information from four microarray studies to build an inter-study validated meta-signature for predicting survival in breast cancer patients.

As described earlier, a common set of 2555 genes was used in this meta-analysis, as it is preferred to provide the same context for data-driven estimation of the posterior probabilities. Functional annotation of the meta-signature revealed genes such as Cyclin E and BCL2, which were previously shown to be correlated with survival outcome in breast cancer (Keyomarsi et al., N Engl J Med 2002, 347:1566-75; O'Driscoll et al., Cancer Lett 2003, 201:225-36). A strength of the inter-study validated signature is the capability of recruiting genes which may not be significant in one study due to limiting sample size or artifacts of the experiments. In this sense, the meta-signature is more stable and less subjective to variations in subsets of the samples. As a result, the predictive genes in a meta-signature may carry more reliable information about tumor progression and patient survival.

In conclusion, a distinction of the analysis presented herein is that the methods of the present invention identified genes that were predictive of recurrence rather than predictive of diseased versus non-diseased status. Given the heterogeneity of the tumors with respect to treatment response and survival outcome, a prognostic prediction analysis is generally more difficult because it is a more complicated phenotype. Further, a prognostic signature (classifier) of failure risk trained in one cohort is often times difficult to validate in independent cohorts. In some embodiments, methods of the present invention provide more powerful gene signatures that are predictive of prognosis because they are validated across multiple studies.

I. Meta-Analysis

As described above, in some embodiments, the present invention provides methods of analyzing multiple expression profile studies to generate meta signatures (e.g., prognostic meta signatures). In some preferred embodiments, the expression profiles compare disease to non-disease states. In some particularly preferred embodiments, the expression profiles compare gene expression in cancerous tissue or cells to normal tissue or cells. The methods and compositions of the present invention thus provide improved methods for the identification of markers across multiple studies.

A. Statistical Methods

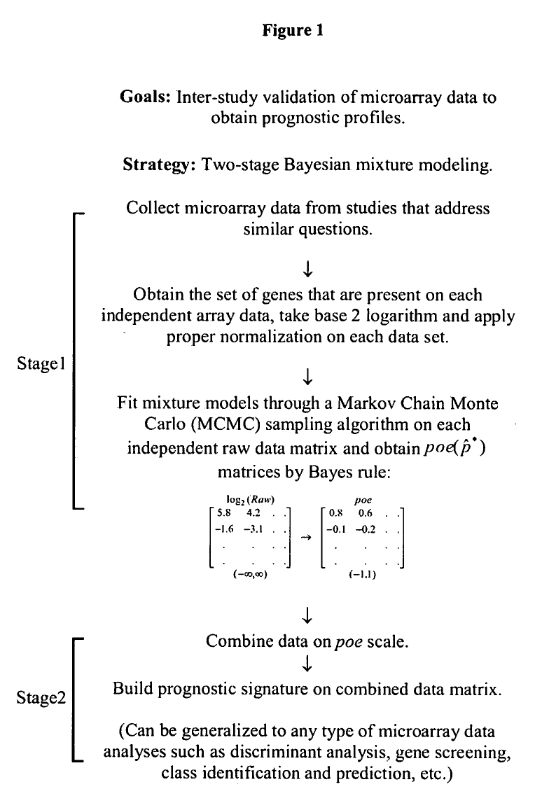

As described above, the present invention provides statistical methods for analyzing multiple expression profile studies. A flow chart of exemplary statistical methods for the meta-analysis of multiple gene expression studies is shown in FIG. 1. In some embodiments, a two stage Bayesian mixture modeling method is employed. For example, in some embodiments, in stage 1, mathematical manipulations of expression data (e.g., base 2 logarithms) is followed by normalization of each data set to be analyzed. Any number of data sets may be utilized (e.g., greater than 2, preferably greater than 3, and even more preferably greater than 5). In some embodiments, the mixture models are fit to a Markov Chain Monte Carlo sampling algorithm (See e.g., Experimental Section below) to obtain probability of expression (poe) matrices by Bayes rule.

In some embodiments, in the second stage of analysis, data is combined on the poe scale to build a prognostic signature. The prognostic signature encompasses all of the combined data of the multiple studies.

The present invention is not limited to the nature of the microarray data. The present invention is illustrated with gene expression data. However, the methods and compositions of the present invention are also suitable for analysis of other types of microarray data including, but not limited to, discriminant analysis, gene screening, and class identification and prediction.

B. Prognostic Signatures

As described above, in some embodiments, the methods of the present invention are utilized to generate meta-signatures (e.g., prognostic signatures) across multiple data sets (e.g., expression profile data sets). The prognostic signatures of the present invention find use in the diagnosis and characterization of disease states (e.g., cancer). In some preferred embodiments, the signatures provide signatures correlation with prognosis (e.g., risk of cancer recurrence or metastasis). For example, in some embodiments, prognostic signatures that represent a prognosis state are compared to a subject's expression profile or other microarray profile to provide a prognosis or diagnosis of a disease state. In other embodiments, prognostic signatures corresponding to a class or grade of disease (e.g., metastatic vs. local tumors) are generated and compared to a subject's expression profile in order to characterize a disease state (e.g., type or grade of cancer). In still further embodiments, prognostic signatures (e.g., of primary vs. metastatic cancer) are used to provide a prognosis to a subject (e.g., primary cancer likely to metastasize vs. primary cancer unlikely to metastasize).

In yet other embodiments, prognostic signatures are used to identify disease (e.g., cancer) markers. The presence of altered expression of a marker across multiple expression profiling studies provides further validation of a marker's disease association. Disease (e.g., cancer) markers identified using the methods of the present invention find use in a variety of further diagnostic and clinical applications (See e.g., below description).

II. Detection of Markers

In some embodiments, the present invention provides methods for detection of expression of disease markers (e.g., cancer markers). In preferred embodiments, expression is measured directly (e.g., at the RNA or protein level). In some embodiments, expression is detected in tissue samples (e.g., biopsy tissue). In other embodiments, expression is detected in bodily fluids (e.g., including but not limited to, plasma, serum, whole blood, mucus, and urine). The present invention further provides panels and kits for the detection of markers. In preferred embodiments, the presence of a cancer marker is used to provide a prognosis to a subject. For example, if a subject is found to have a marker indicative of a highly metastasizing tumor, additional therapies (e.g., hormonal, chemotherapy or radiation therapies) can be started at a earlier point when they are more likely to be effective (e.g., before metastasis). In addition, if a subject is found to have a tumor that is not responsive to a given therapy, the expense and inconvenience of such therapies can be avoided.

The present invention is not limited to a particular disease marker. Experiments conducted during the course of development of the present invention identified breast cancer markers. However, additional disease markers are also contemplated to be within the scope of the present invention. 1. Detection of RNA

In some embodiments, detection of disease markers (e.g., including but not limited to, those disclosed herein) is detected by measuring the expression of corresponding mRNA in a tissue sample. mRNA expression may be measured by any suitable method, including but not limited to, those disclosed below.

In some embodiments, RNA is detection by Northern blot analysis. Northern blot analysis involves the separation of RNA and hybridization of a complementary labeled probe. An exemplary method for Northern blot analysis is provided in Example 3.

In other embodiments, RNA expression is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific nucleic acid (e.g., RNA) sequences by using structure-specific enzymes to cleave a complex formed by the hybridization of overlapping oligonucleotide probes.

In still further embodiments, RNA (or corresponding cDNA) is detected by hybridization to a oligonucleotide probe). A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

2. Detection of Protein

In other embodiments, gene expression of disease markers is detected by measuring the expression of the corresponding protein or polypeptide. Protein expression may be detected by any suitable method. In some embodiments, proteins are detected by their binding to an antibody raised against the protein (e.g., using immunohistochemistry techniques). The generation of antibodies is described below.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized.

In other embodiments, the immunoassay described in U.S. Pat. Nos. 5,599,677 and 5,672,480; each of which is herein incorporated by reference.

3. Kits

In yet other embodiments, the present invention provides kits for the detection of expression of disease markers. In some embodiments, the kits contain antibodies specific for a cancer marker, in addition to detection reagents and buffers. In other embodiments, the kits contain reagents specific for the detection of mRNA or cDNA (e.g., oligonucleotide probes or primers). In preferred embodiments, the kits contain all of the components necessary to perform a detection assay, including all controls, directions for performing assays, and any necessary software for analysis and presentation of results.

4. In Vivo Imaging

In some embodiments, in vivo imaging techniques are used to visualize the expression of disease markers in an animal (e.g., a human or non-human mammal). For example, in some embodiments, cancer marker mRNA or protein is labeled using an labeled antibody specific for the disease marker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the disease markers of the present invention are described below.

The in vivo imaging methods of the present invention are useful in the diagnosis of tissues that express the markers of the present invention (e.g., cancer). In vivo imaging is used to visualize the presence of a marker indicative of the disease. Such techniques allow for diagnosis without the use of an unpleasant biopsy or other invasive diagnostic technique. The in vivo imaging methods of the present invention are also useful for providing prognoses to patients. For example, the presence of a marker indicative of cancers likely to metastasize can be detected. The in vivo imaging methods of the present invention can further be used to detect metastatic cancers in other parts of the body.

In some embodiments, reagents (e.g., antibodies) specific for the disease markers of the present invention are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 [1990] have described an optimized antibody-chelator for the radioimmunoscintographic imaging of tumors using Indium-111 as the label. Griffin et al., (J Clin Onc 9:631-640 [1991]) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 [1991]). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 [1980]) for In-111 and Tc-99m, and by Scheinberg et al. (Science 215:1511 [1982]). Other chelating agents may also be used, but the 1-(p-carboxymethoxybenzyl)EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 [1982]) for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

A preferred method of labeling immunoglobulins with Tc-99m is that described by Wong et al. (Int. J. Appl. Radiat. Isot., 29:251 [1978]) for plasma protein, and recently applied successfully by Wong et al. (J. Nucl. Med., 23:229 [1981]) for labeling antibodies.

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the specific cancer marker of the present invention, to insure that the antigen binding site on the antibody will be protected. The antigen is separated after labeling.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a marker of the present invention). When active, it leads to a reaction that emits light. A CCD camera and software is used to capture the image and analyze it.

III. Antibodies

The present invention provides isolated antibodies. In preferred embodiments, the present invention provides monoclonal antibodies that specifically bind to an isolated polypeptide comprised of at least five amino acid residues of the disease markers identified using the methods of the present invention. These antibodies find use in the diagnostic and research methods described herein.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a cancer marker of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and, the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a disease marker of the present invention (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

IV. Drug Screening

In some embodiments, the present invention provides drug screening assays (e.g., to screen for anticancer drugs). The screening methods of the present invention utilize cancer markers identified using the methods of the present invention. For example, in some embodiments, the present invention provides methods of screening for compound that alter (e.g., increase or decrease) the expression of disease marker genes. In some embodiments, candidate compounds are antisense agents (e.g., oligonucleotides) directed against disease markers. See Section V below for a discussion of antisense therapy. In other embodiments, candidate compounds are antibodies that specifically bind to a disease marker of the present invention.

In one screening method, candidate compounds are evaluated for their ability to alter disease marker expression by contacting a compound with a cell expressing a disease marker and then assaying for the effect of the candidate compounds on expression. In some embodiments, the effect of candidate compounds on expression of a disease marker gene is assayed for by detecting the level of disease marker mRNA expressed by the cell. mRNA expression can be detected by any suitable method. In other embodiments, the effect of candidate compounds on expression of disease marker genes is assayed by measuring the level of polypeptide encoded by the disease markers. The level of polypeptide expressed can be measured using any suitable method, including but not limited to, those disclosed herein.

In other embodiments, the effect of a candidate compound on a subject's expression profile is analyzed. The subject's expression profile is compared to a meta-signature (e.g., prognostic meta-signature). The effect of the compound on prognosis can thus be determined.

Specifically, the present invention provides screening methods for identifying modulators, i.e., candidate or test compounds or agents (e.g., proteins, peptides, peptidomimetics, peptoids, small molecules or other drugs) which bind to disease markers of the present invention, have an inhibitory (or stimulatory) effect on, for example, cancer marker expression or cancer markers activity, or have a stimulatory or inhibitory effect on, for example, the expression or activity of a cancer marker substrate. Compounds thus identified can be used to modulate the activity of target gene products (e.g., disease marker genes) either directly or indirectly in a therapeutic protocol, to elaborate the biological function of the target gene product, or to identify compounds that disrupt normal target gene interactions. In some embodiments, compounds that inhibit the activity or expression of disease markers find use in the treatment of proliferative disorders, e.g., cancer.

In one embodiment, the invention provides assays for screening candidate or test compounds that are substrates of a disease marker protein or polypeptide or a biologically active portion thereof. In another embodiment, the invention provides assays for screening candidate or test compounds that bind to or modulate the activity of a disease marker protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. NatI. Acad. Sci. 87:6378-6382 [1990]; Felici, J. Mol. Biol. 222:301 [1991]).

In one embodiment, an assay is a cell-based assay in which a cell that expresses a disease marker protein or biologically active portion thereof is contacted with a test compound, and the ability of the test compound to the modulate disease marker activity is determined. Determining the ability of the test compound to modulate disease marker activity can be accomplished by monitoring, for example, changes in enzymatic activity. The cell, for example, can be of mammalian origin.

The ability of the test compound to modulate disease marker binding to a compound, e.g., a disease marker substrate, can also be evaluated. This can be accomplished, for example, by coupling the compound, e.g., the substrate, with a radioisotope or enzymatic label such that binding of the compound, e.g., the substrate, to a cancer marker can be determined by detecting the labeled compound, e.g., substrate, in a complex.

Alternatively, the disease marker is coupled with a radioisotope or enzymatic label to monitor the ability of a test compound to modulate disease marker binding to a disease marker substrate in a complex. For example, compounds (e.g., substrates) can be labeled with ¹²⁵I, ³⁵S ¹⁴C or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a disease marker substrate or signaling molecule) to interact with a disease marker with or without the labeling of any of the interactants can be evaluated. For example, a microphysiorneter can be used to detect the interaction of a compound with a disease marker without the labeling of either the compound or the disease marker (McConnell et al. Science 257:1906-1912 [1992]). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and disease markers.

In yet another embodiment, a cell-free assay is provided in which a disease marker protein or biologically active portion thereof is contacted with a test compound and the ability of the test compound to bind to the disease marker protein or biologically active portion thereof is evaluated. Preferred biologically active portions of the disease markers proteins to be used in assays of the present invention include fragments that participate in interactions with substrates or other proteins, e.g., fragments with high surface probability scores.

Cell-free assays involve preparing a reaction mixture of the target disease marker protein and the test compound under conditions and for a time sufficient to allow the two components to interact and bind, thus forming a complex that can be removed and/or detected.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in 1 5 the assay should be maximal. An FRET binding event can be conveniently measured through standard fluorometric detection means well known in the art (e.g., using a fluorimeter).

In another embodiment, determining the ability of the disease marker protein to bind to a target molecule can be accomplished using real-time Biomolecular Interaction Analysis (BIA) (see, e.g., Sjolander and Urbaniczky, Anal. Chem. 63:2338-2345 [1991] and Szabo et al. Curr. Opin. Struct. Biol. 5:699-705 [1995]). “Surface plasmon resonance” or “BIA” detects biospecific interactions in real time, without labeling any of the interactants (e.g., BlAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize disease markers, an anti-disease marker antibody or its target molecule to facilitate separation of complexed from non-complexed forms of one or both of the proteins, as well as to accommodate automation of the assay. Binding of a test compound to a disease marker protein, or interaction of a disease marker protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-disease marker fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or disease marker protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Alternatively, the complexes can be dissociated from the matrix, and the level of cancer markers binding or activity determined using standard techniques. Other techniques for immobilizing either disease marker protein or a target molecule on matrices include using conjugation of biotin and streptavidin. Biotinylated disease marker protein or target molecules can be prepared from biotin-NHS (N-hydroxy-succinimide) using techniques known in the art (e.g., biotinylation-kit, Pierce Chemicals, Rockford, EL), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-IgG antibody).

This assay is performed utilizing antibodies reactive with disease marker protein or target molecules but which do not interfere with binding of the disease marker protein to its target molecule. Such antibodies can be derivatized to the wells of the plate, and unbound target or disease marker protein trapped in the wells by antibody conjugation. Methods for detecting such complexes, in addition to those described above for the GST-immobilized complexes, include immunodetection of complexes using antibodies reactive with the disease marker protein or target molecule, as well as enzyme-linked assays which rely on detecting an enzymatic activity associated with the disease marker protein or target molecule.

Alternatively, cell free assays can be conducted in a liquid phase. In such an assay, the reaction products are separated from unreacted components, by any of a number of standard techniques, including, but not limited to: differential centrifugation (see, for example, Rivas and Minton, Trends Biochem Sci 18:284-7 [1993]); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology 1999, J. Wiley: New York). Such resins and chromatographic techniques are known to one skilled in the art (See e.g., Heegaard J. Mol. Recognit 11:141-8 [1998]; Hageand Tweed J. Chromatogr. Biomed. Sci. Appl 699:499-525 [1997]). Further, fluorescence energy transfer may also be conveniently utilized, as described herein, to detect binding without further purification of the complex from solution.

The assay can include contacting the disease marker protein or biologically active portion thereof with a known compound that binds the disease marker to form an assay mixture, contacting the assay mixture with a test compound, and determining the ability of the test compound to interact with a disease marker protein, wherein determining the ability of the test compound to interact with a disease marker protein includes determining the ability of the test compound to preferentially bind to disease markers or biologically active portion thereof, or to modulate the activity of a target molecule, as compared to the known compound.

To the extent that disease markers can, in vivo, interact with one or more cellular or extracellular macromolecules, such as proteins, inhibitors of such an interaction are useful. A homogeneous assay can be used can be used to identify inhibitors.

For example, a preformed complex of the target gene product and the interactive cellular or extracellular binding partner product is prepared such that either the target gene products or their binding partners are labeled, but the signal generated by the label is quenched due to complex formation (see, e.g., U.S. Pat. No. 4,109,496, herein incorporated by reference, that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, disease markers protein can be used as a “bait protein” in a two-hybrid assay or three-hybrid assay (see, e.g., U.S. Pat. No. 5,283,317; Zervos et al., Cell 72:223-232 [1993]; Madura et al., J. Biol. Chem. 268.12046-12054 [1993]; Bartel et al., Biotechniques 14:920-924 [1993]; Iwabuchi et al., Oncogene 8:1693-1696 [1993]; and Brent W0 94/10300; each of which is herein incorporated by reference), to identify other proteins, that bind to or interact with disease markers (“disease marker-binding proteins” or “disease marker-bp”) and are involved in disease marker activity. Such disease marker-bps can be activators or inhibitors of signals by the disease marker proteins or targets as, for example, downstream elements of a disease markers-mediated signaling pathway.

Modulators of disease markers expression can also be identified. For example, a cell or cell free mixture is contacted with a candidate compound and the expression of disease marker mRNA or protein evaluated relative to the level of expression of disease marker mRNA or protein in the absence of the candidate compound. When expression of disease marker mRNA or protein is greater in the presence of the candidate compound than in its absence, the candidate compound is identified as a stimulator of disease marker mRNA or protein expression. Alternatively, when expression of disease marker mRNA or protein is less (i.e., statistically significantly less) in the presence of the candidate compound than in its absence, the candidate compound is identified as an inhibitor of disease marker mRNA or protein expression. The level of disease markers mRNA or protein expression can be determined by methods described herein for detecting disease marker mRNA or protein.

A modulating agent can be identified using a cell-based or a cell free assay, and the ability of the agent to modulate the activity of a disease markers protein can be confirmed in vivo, e.g., in an animal such as an animal model for a disease (e.g., an animal with cancer or metastatic cancer; or an animal harboring a xenograft of a prostate cancer from an animal (e.g., human) or cells from a cancer resulting from metastasis of a cancer (e.g., to a lymph node, bone, or liver), or cells from a prostate cell line.

This invention further pertains to novel agents identified by the above-described screening assays (See e.g., below description of disease therapies). Accordingly, it is within the scope of this invention to further use an agent identified as described herein (e.g., a disease marker modulating agent, an antisense disease marker nucleic acid molecule, a siRNA molecule, a disease marker specific antibody, or a disease marker-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

IV. Disease Therapies

In some embodiments, the present invention provides therapies for diseases characterized by altered expression of disease markers identified using the methods of the present invention. Any disease or altered state characterized by aberrant expression of a disease marker (e.g., cancer) may be treated using the below described methods.

A. Antisense Therapies

In some embodiments, the present invention targets the expression of disease markers. For example, in some embodiments, the present invention employs compositions comprising oligomeric antisense compounds, particularly oligonucleotides (e.g., those identified in the drug screening methods described above), for use in modulating the function of nucleic acid molecules encoding disease markers of the present invention, ultimately modulating the amount of disease marker expressed. This is accomplished by providing antisense compounds that specifically hybridize with one or more nucleic acids encoding disease markers of the present invention. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of disease markers of the present invention. In the context of the present invention, “modulation” means either an increase (stimulation) or a decrease (inhibition) in the expression of a gene. For example, expression may be inhibited to potentially prevent tumor proliferation.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a disease marker of the present invention. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in U.S. patent WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos.: 5,539,082; 5,714,331; and 5,719,262, each of which is herein incorporated by reference. Further teaching of PNA compounds can be found in Nielsen et al., Science 254:1497 (1991).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further preferred modification includes 2′-dimethylaminooxyethoxy (i.e., a O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy(2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2. degree °C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-S-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisensce oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” anti sense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above.

The present invention also includes pharmaceutical compositions and formulations that include the antisense compounds of the present invention as described below.

B. RNA Interference (RNAi)

In some embodiments, RNAi is utilized to inhibit disease marker function. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC (RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments.

Chemically synthesized siRNAs have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67, herein incorporated by reference).

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001;15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

C. Genetic Therapies

The present invention contemplates the use of any genetic manipulation for use in modulating the expression of disease markers of the present invention. Examples of genetic manipulation include, but are not limited to, gene knockout (e.g., removing the disease marker gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

D. Antibody Therapy

In some embodiments, the present invention provides antibodies that target prostate tumors that express a disease marker of the present invention. Any suitable antibody (e.g., monoclonal, polyclonal, or synthetic) may be utilized in the therapeutic methods disclosed herein. In preferred embodiments, the antibodies used for disease therapy are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the therapeutic antibodies comprise an antibody generated against a disease marker of the present invention, wherein the antibody is conjugated to a cytotoxic agent. In such embodiments, a tumor specific therapeutic agent is generated that does not target normal cells, thus reducing many of the detrimental side effects of traditional chemotherapy. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of endothelial cells. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention may include a variety of cytotoxic moieties, including but not limited to, radioactive isotopes (e.g., iodine-131, iodine-123, technicium-99m, indium-Il1, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments may include agents such as a coagulant, a cytokine, growth factor, bacterial endotoxin or the lipid A moiety of bacterial endotoxin. For example, in some embodiments, therapeutic agents will include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some preferred embodiments, deglycosylated ricin A chain is utilized.

In any event, it is proposed that agents such as these may, if desired, be successfully conjugated to an antibody, in a manner that will allow their targeting, internalization, release or presentation to blood components at the site of the targeted disease (e.g., tumor) cells as required using known conjugation technology (See, e.g., Ghose et al., Methods Enzymol., 93:280 [1983]).

For example, in some embodiments the present invention provides immunotoxins targeted against a disease marker of the present invention. Immunotoxins are conjugates of a specific targeting agent typically a tumor-directed antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In other embodiments, particularly those involving treatment of solid tumors, antibodies are designed to have a cytotoxic or otherwise anticellular effect against the tumor vasculature, by suppressing the growth or cell division of the vascular endothelial cells. This attack is intended to lead to a tumor-localized vascular collapse, depriving the tumor cells, particularly those tumor cells distal of the vasculature, of oxygen and nutrients, ultimately leading to cell death and tumor necrosis.

In preferred embodiments, antibody based therapeutics are formulated as pharmaceutical compositions as described below. In preferred embodiments, administration of an antibody composition of the present invention results in a measurable decrease in disease (e.g., decrease or elimination of tumor).

E. Pharmaceutical Compositions

The present invention further provides pharmaceutical compositions (e.g., comprising the therapeutic compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parentefal, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), NEOPHECTIN (available from NeoPhram), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the compounds of the formulation.

Certain embodiments of the invention provide pharmaceutical compositions containing (a) one or more compounds of the present invention and (b) one or more other chemotherapeutic agents that function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Other non-antisense chemotherapeutic agents are also within the scope of this invention. Two or more combined compounds may be used together or sequentially.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compound is administered in maintenance doses, ranging from 0.01 μg to 100 g per kg of body weight, once or more daily, to once every 20 years.

VI. Transgenic Animals Expressing Disease Marker Genes

The present invention contemplates the generation of transgenic animals comprising an exogenous disease marker gene of the present invention or mutants and variants thereof (e.g., truncations or single nucleotide polymorphisms). In preferred embodiments, the transgenic animal displays an altered phenotype (e.g., increased or decreased presence of markers) as compared to wild-type animals. Methods for analyzing the presence or absence of such phenotypes include but are not limited to, those disclosed herein. In some preferred embodiments, the transgenic animals further display an increased or decreased growth of disease (e.g., tumors or evidence of cancer).

The transgenic animals of the present invention find use in drug (e.g., cancer therapy) screens. In some embodiments, test compounds (e.g., a drug that is suspected of being useful to treat cancer) and control compounds (e.g., a placebo) are administered to the transgenic animals and the control animals and the effects evaluated.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad Sci. USA 82:6927 [1985]). Transfection is easily and efficiently obtained by culturing the blastomeres on a monolayer of virus-producing cells (Stewart, et al., EMBO J., 6:383 [1987]). Alternatively, infection can be performed at a later stage. Virus or virus-producing cells can be injected into the blastocoele (Jahner et al., Nature 298:623 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized to knock-out gene function or create deletion mutants (e.g., truncation mutants). Methods for homologous recombination are described in U.S. Pat. No. 5,614,396, incorporated herein by reference.

Experimental

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); and ° C. (degrees Centigrade).

A. Methods

Data collection and preparation. The breast cancer microarray data sets were obtained at the author's websites from four recently published studies (Sorlie et al., Proc Natl Acad Sci 2001, 98:10869-74; van't Veer et al., Nature 2002, 415:530-6; Sotiriou et al., Proc Natl Acad Sci 2003, 100:10393-8; Huang et al., Lancet 2003, 361:1590-6). Each data were preprocessed, either by a lowess normalization for two-channel microarray data (Yang et al., Nucleic Acids Research 2002, 30:e15) or a robust analysis for Affymetrix data (Irizarry et al., Biostatistics 2003, 4:249-64). Data sets were filtered for a common set of 2,555 genes from these four studies by Unigene Cluster IDs. Each data matrix of the 2,555 genes was then normalized by median centering and dividing by the standard deviation for each gene. Missing data were imputed by the k-nearest neighbors imputation algorithm (Troyanskaya et al., Bioinformatics 2001, 17:520).
Mixture modeling of microarray data. Each of the four raw data sets was treated as an expression matrix X with elements Xᵢⱼ, where i=1, . . . , mk, j=1, . . . , n (k=1, . . . , 4 and n=2,555). The expression measurement Xᵢⱼ can be the ratio of the two fluorescent dye hybridization intensities for the spotted cDNA arrays (Sorlie et al., supra; Sotiriou et al., supra) and the Intjek oligonucleotide array (van't Veer et al., supra), or averaged difference between the perfect match and mismatch probe hybridizations for the Affymetrix gene chip (Huang et al., supra). Let E be a latent class variable, and eᵢⱼ indicates over-, under- or normal expression for each entry of the R matrices. Then:

$${e}_{ij} = \{ \begin{matrix}1 & gene j is overexpressed in sample i ; \\ 0 & gene j is normally expressed in sample i ; \\ - 1 & gene j is underexpressed in sample i .\end{matrix}$$

The values of eᵢⱼ are latent and not directly observed from the data. The probabilities of eᵢⱼ being 1 or −1 were estimated given the observed raw expression xᵢⱼ, which were denoted as
pᵢⱼ⁺=Pr(eᵢⱼ=1|xᵢⱼ) and (pᵢⱼ⁻=Pr(eᵢⱼ=−1|xᵢⱼ).
Estimates of these latent quantities were obtained under a Bayesian mixture model setting. In particular, it is assumed that the raw expression xᵢⱼ falls into one of the three expression categories. For each gene j, the expression then arises from a mixture of three distributions:
(xᵢⱼ|eᵢⱼ=1)˜ƒ_{1,j}(·), (xᵢⱼ|eᵢⱼ=0)˜ƒ_{0,j}(·), and (xᵢⱼ|eᵢⱼ=−1)˜ƒ_{-1,j}(·).
In the mixture, ƒ₁ⱼ, ƒ₀ⱼ and ƒ₋₁ⱼ are the density functions of the following distributions:
U(αᵢ+μⱼ,αᵢ+μⱼ+κⱼ⁺), N(αᵢ+μⱼ,σⱼ²), and U(−κⱼ⁻+αᵢ+μⱼ,αᵢ+μⱼ),
respectively. Here, U refers to a uniform distribution and N refers to a normal distribution. αᵢ+μⱼ is both the mean of the normal distribution and the threshold point in the uniform distribution. μⱼ is the gene effect and αᵢ is the sample effect. The κⱼ⁺ and κⱼ⁻ provide limits to the uniform distribution in the mixture, and are set to be at least 3 σ.
πⱼ⁺=P(eᵢⱼ=1) and πⱼ⁻=Peᵢⱼ=−1)
are the multinomial probabilities for eᵢⱼ. With the specifications of models, the latent quantities can be calculated by Bayes' rule:

$${p}_{ij}^{+} = P ( {e}_{ij} = 1 \vert {x}_{ij} ) = \frac{{π}_{j}^{+} {f}_{1 , j} ( {x}_{ij} )}{{π}_{j}^{+} {f}_{1 , j} ( {x}_{ij} ) + {π}_{j}^{-} {f}_{- 1 , j} ( {x}_{ij} ) + ( 1 - {π}_{j}^{+} - {π}_{j}^{-} ) {f}_{0 , j} ( {x}_{ij} )}$$

$${p}_{ij}^{-} = P ( {e}_{ij} = - 1 \vert {x}_{ij} ) = \frac{{π}_{j}^{-} {f}_{- 1 , j} ( {x}_{ij} )}{{π}_{j}^{+} {f}_{1 , j} ( {x}_{ij} ) + {π}_{j}^{-} {f}_{- 1 , j} ( {x}_{ij} ) + ( 1 - {π}_{j}^{+} - {π}_{j}^{-} ) {f}_{0 , j} ( {x}_{ij} )} .$$

By noting that the supports for the two uniform distributions are disjoint, the probabilities of differential expression are mutually exclusive with the forms:

$$( {p}^{+} , {p}^{-} ) = ( \frac{{π}^{+} / {k}^{+}}{{π}^{+} / {κ}^{+} + ( 1 - {π}_{j}^{+} - {π}_{j}^{-} ) {f}_{0}} , 0 )$$

$$or \\ ( {p}^{+} , {p}^{-} ) = ( 0 , \frac{{π}^{-} / {k}^{-}}{{π}^{-} / {κ}^{-} + ( 1 - {π}_{j}^{+} - {π}_{j}^{-} ) {f}_{0}} ) .$$

A one dimension measure can thus be constructed as poe=p⁺−p⁻. As a result, poe ranges from −1 to 1, and can be interpreted as the signed conditional probability of differential expression.

To borrow strength across genes, the estimation of the gene-specific parameters was formulated under a Bayesian hierarchical model setting. Given the large amount of parameters, prior distributions were specified to model the variation of the gene-specific parameter estimates, in particular,
μⱼ˜N(θ_{μ},τ_{μ}); κⱼ⁺˜Exp(θ_{κ}⁺); logit(πⱼ⁺)˜N(θ_{π}^{+,τ}_{π}⁺);
σⱼ⁻²˜Gamma(r,λ); κⱼ⁻˜Exp(θ_{κ}⁻); logit(πⱼ⁻)˜N(θ_{π}⁻,τ_{π}⁻).
The recommendations of Parmigiani et al. (Parmigiani et al., J R Stat Soc B 2002, 64:717-36) were followed in terms of the prior choices. A Metropolis-Hastings MCMC sampling algorithm was then implemented to approximate the posterior distributions of the parameters. Data augmentation started at a set of data-driven initiating parameter values. For example, trimmed means and variances across samples were used as the initial values for the parameters in the normal distribution of the mixture. Further details of the Bayesian hierarchical mixture model can be found in Parmigiani et al. (supra).

Matrices of
poe(p*=p⁺−p⁻)
were obtained for each of the five data sets.
Leave-one-out cross validation and risk index computation. For the combined sample pool of the breast cancer patients (the meta-cohort), outcome groups were defined as recurred during followup and remained relapse-free for at least 3 years. In particular, Let Tᵢ be the event time for subject i, Cᵢ be the censoring time for subject i, and δᵢ=1{Tᵢ<Cᵢ} be the censoring indicator. Define a new outcome variable,

$${y}_{i} = \{ \begin{matrix}1 , & {δ}_{i} = 1 \\ 0 , & {δ}_{i} = 0 and {C}_{i} \geq {t}^{*}\end{matrix} ,$$

where t* can be specified with clinical knowledge. t*=3 years was chosen in this study. Constructing classifiers were then considered using y; note that y=1 corresponds to the poor outcome group and y=0 to the good outcome group. The sample sizes for each study are shown in Table 1.

Logistic regression was used to build a classifier for prognosis. For each gene j, the following univariate logistic regression model was fit using data from all studies:
logit{Pr(yᵢ=1|xᵢⱼ^{*})}=ηⱼ+βⱼxᵢⱼ^{*},
where x* is the rescaled value that allows data integration across multiple studies. The estimated values of βⱼ, \hat{β}ⱼ, are then used to form a risk score using a variation of the compound covariate predictor method (Tukey et al., Control Clin Trials 1993, 14:266-85; Radmacher et al., J Comput Biol 2002, 9:505-11); for a given set of covariate values x₁, . . . , x_{D}, the risk index is given as

$$RI = {\sum }_{j = 1}^{D} {\hat{β}}_{j} {x}_{j} .$$

To assess the performance of the classifier, the effect of training and testing the model using the same data was addressed. An “honest” estimate of the prediction error rate is obtained using leave-one-out cross-validation. Define a risk index

$$RI = {\sum }_{j = 1}^{D} {\hat{β}}_{j , - i} {x}_{ij}^{*} , \\ where$$

$$i = 1 , … , {\sum }_{i = 1}^{K} {m}_{k} , and {\hat{β}}_{j , - i}$$

is the effect estimate for gene j in the combined meta-cohort without the i^{th} sample. The risk index for sample i is a weighted linear combination of the expression profiles of the top D genes, where the ranking of the genes is based on their corresponding significance in the univariate logistic model fit. Classification of sample i to the risk groups is then based on the ith leave one-out risk index, i.e., C(X*)=I{RIᵢ>c} with c being the empirical quantiles. (40th -70th) of the RI's. The number of genes D in a classifier is treated as a parameter and optimized to minimize the prediction error rates.

The list of the top cumulative genes in the meta-signature was obtained by ranking all 2,555 genes by the number of times in the leave-one-out cross-validation steps that each one had a P-value from the univariate logistic regression less than 0.001.

Heat map display. The treeview software was used (Eisen et al., Proc Natl Acad Sci 1998, 95:14863-8) to generate a heat map representation of the poe pro-files of the meta-signature. Yellow represents high probability of over-expression and blue represents high probability of under-expression. For heat maps of raw data matrices, the data was preprocessed by mean centering and then dividing by the standard deviation for each row. The means and the standard deviations used in the normalization were the relapse-free (RF) sample means and variances for each study data. The values for the recurrence (R) samples after standardizing then represented the number of standard deviations over or under the mean RF sample expression.

B. Results

Development of the two-stage Bayesian mixture modeling approach for the meta-analysis of microarray data. FIG. 1 outlines the two-stage Bayesian mixture modeling strategy. The strategy was used to build a scale that can be combined across different microarray platforms, and therefore allows simultaneous examination of independent data sets. The stage 1 of the analysis involves data-driven estimation of posterior probability of differential expression, namely poe. The Bayesian hierarchical model employed for estimation borrows strength across genes by assuming further distributions for the gene-specific parameters (see Methods). For data integration purposes, a common set of 2,555 genes that were profiled in each of the four studies was utilized. Although the cost for compiling common genes is a loss of potential predictive features, it is not unreasonable to assume, given the analogous hypothesis explored in each study, that the common set represents the most relevant genes of interest for breast cancer prognosis. The resulting values of poe represent signed probability of differential expression for gene j in sample i, and thus provide a unified measure across studies. Further, the transformation improves contrast in each data set by removing the influence of extreme expression values. In stage 2, the expression profile of tumor samples from multiple studies were combined on the poe scale to generate a meta-cohort. The benefit of data integration using poe is twofold. First, it improves power of statistical analysis by increasing the sample size. Such integration of independent data sets renders sensitivity to those small yet consistent expression changes for certain genes. Second, it reduces the chance of false positive features due to artifacts from a single study, and allows reliable findings across studies. This Example integrated four breast cancer microarray data sets of distinct platforms (Table 1), and developed a prognostic meta-signature for disease recurrence.

Building a gene expression meta-signature for breast cancer prognosis. In the second stage of the analysis, the performance of the genes found using the meta-analysis methods was assessed based on classification accuracy. The response that the present example builds classifiers to predict is time to breast cancer recurrence. While the ideal data would have information on time to recurrence on all subjects (potentially censored), not all studies have the time to recurrence information available and instead provide data on recurrence within a certain time interval (e.g., recurrence within five years versus no recurrence within five years). To deal with this issue, a dichotomization was utilized where a bad outcome is recurrence during follow-up and a good outcome is remaining recurrence-free for at least three years. The additional constraint for the good outcome group is to reduce potential bias introduced by short censoring due to insufficient length of follow-up. This is particularly relevant in cross-study analysis, given the heterogeneity in patient recruitment criteria and study designs. Accordingly, of the combined meta-cohort (n=305) of breast cancer patients, 48.9% were in the poor outcome group, whereas 51.1% in the good outcome group. The sample sizes for each study are shown in Table 1. Each gene was then associated with the recurrence status by a logistic regression within a leave-one-out cross validation scheme, and rank-ordered by the significance level of the coefficient. As a result, 23 genes held up as significant predictor of recurrence (P≦0.001) in all cross-validation steps, representing a cohort of essential genes strongly associated with breast cancer recurrence. By random chance, there would be on average 2.5 genes to be found significant at P≦0.001 in a set of 2,555. By finding 23 genes with a P≦0.001, it is clear that there are much more predictive features than would be expected by random chance.

To identify a prognostic meta-signature, a risk index (RI) was defined as a linear combination of the poe profile and the coefficient estimates from the univariate logistic regression for each gene j. Large positive values of RI indicate high risk of failure, whereas large negative values of RI indicate low risk of failure. Classification of sample i to the risk groups is then based on the ith leave-one-out risk index. The classifier is =I{RIi>c}, with c being the empirical quantiles of the risk indices. The number of genes in a classifier is treated as a parameter and optimized to minimize the prediction error rates.

The 90-gene expression meta-signature predicts clinical outcome in breast cancer patients. By minimizing the misclassification error, a 90 gene meta-signature was obtained that reliably predicts outcome in the meta-cohort. This meta-signature classified 122 patients into a high risk group, where 84 (69%) of them had a recurrence. On the other hand, the signature classified 183 patients into a low risk group, where 118 (64%) of them did not recur by the end of the follow up. By cross tabulating the risk groups predicted by the meta-signature and the actual recurrence status, an estimated odds ratio of 4.0 (95% CI: 2.5-6.5, P<0.0001) was obtained. In spite of the heterogeneity of the combined patient population, the meta-signature predicted the odds of recurrence for a patient showing a high risk signature as four times of the odds of recurrence for a patient showing a low risk signature.

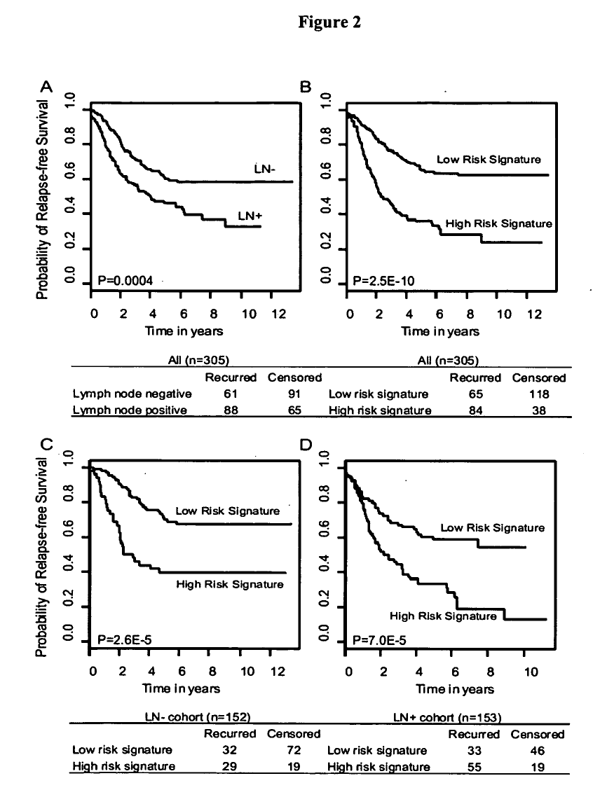

Several studies have implicated that the lymph node status is one of the principal clinical factors to classify patients in relation to the risk of relapse of breast cancer (Carter et al., Cancer 1989, 63:181-187; Fisher et al., Surg Gynecol Obstet 1970, 131:79-88; Smith et.al., Cancer 1977, 39:527-32). Although there have been controversial findings with regard to its predictive values in breast cancer survival outcome, it was shown in the meta-cohort that the nodal status is a significant risk factor of recurrence. The estimated odds of recurrence for node-positive patients is two times higher than the odds of recurrence for node negative patients (95% CI:1.3-3.2, P=0.002) in the combined samples. Kaplan-Meier analysis provides further evidence that the meta-signature was a significant prognostic index of breast cancer recurrence in the meta-cohort (FIG. 2). The estimated three-year survival rate was 76.0%(±3.2%) for low risk signature and 45.9%(±4.5%) for high risk signature. Nodal status, on the other hand, was less discriminative at the three-year time point with an estimated survival rate of 71.7%(±3.7%) for lymph node negative patients and 56.2%(±4.0) for lymph node positive patients. Node-negative patients, although generally considered to be at low risk of recurrence, are heterogeneous in disease progression. About one third of node-negative patients develop local recurrence (Quiet et al., J Clin Oncol 1995, 13:1144-51). Many studies have therefore explored the potential of using molecular biomarkers to further differentiate patient survival outcome in nodal negative cohort (Fioravanti et al., Int J Cancer 1997, 74:620-24; Malley et al., Hum Pathol 1996, 27:655-63; Patel J Surg Oncol 1996, 62:86-92; Reed et al., Cancer 2000, 88:804-13). As shown in FIGS. 2C and 2D, the meta-signature further differentiated 48 (31.6%) of the LN− patients to be at higher risk of recurrence during followup (P<0.0001). Similarly for nodal positive patients, a cohort thought to be at high risk of recurrence, the meta-signature identified 79 (51.6%) of the LN+ patients to have, in fact, lower recurrence risk over time (P<0.0001, FIG. 2D). Nodal status failed to maintain its predictive power after controlling for the meta-signature risk groups (P=0.05 and 0.12 in low risk signature and high risk signature group respectively). A multivariate logistic regression model suggested that the meta-signature is an independent predictor of the recurrent status with respect to nodal status in the metacohort (OR=3.7(2.3-6.1), P<0.0001).

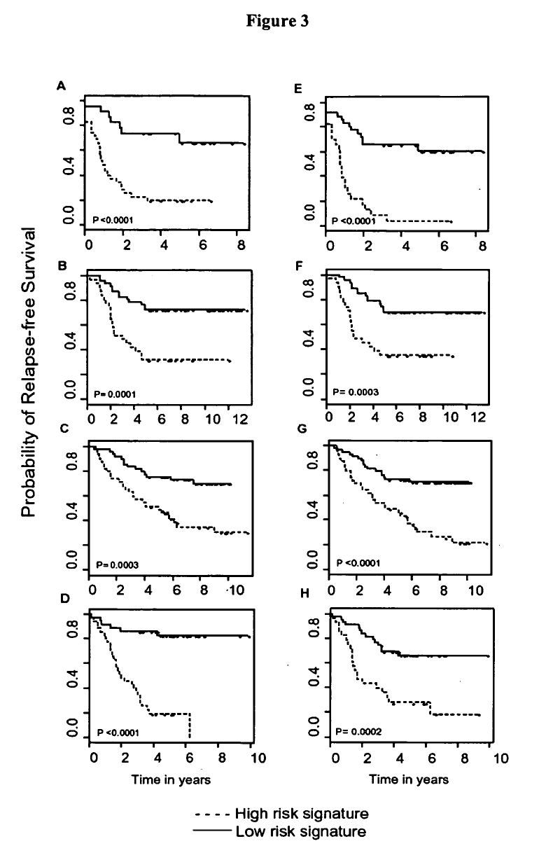

Comparison of the meta-signature to the study-specific signatures. To comprehend the potential gains of such two-stage meta analysis over individual analysis in each single study cohort, study-wise gene expression signatures were constructed using the same method. By minimizing the misclassification errors, a signature consisting of 10, 60, 100, and 130 genes for Sorlie, van't Veer, Sotiriou, and Huang study cohort respectively. The results of the classifiers are summarized in Table 2. Not only did the size of the study-specific signatures vary significantly, the elements of the signatures had very little overlap. At most two genes appeared in more than one signature among the four. In addition, signatures identified in one study tended to have poor performance in other studies. Table 3 lists the estimated odds ratios for disease outcome and risk groups predicted by a gene expression signature. An individual signature identified in one study cohort demonstrated considerable shrinkage in the odds ratio estimates and non-significant 95% confidence intervals in the validation studies, indicating significantly reduced discriminative power in the testing cohorts. Kaplan-Meier analysis provided further evidence that the study-specific signatures performed poorly in pairwise cross-validations. Meta-analysis accounts for such heterogeneity of the individual signatures in two ways. First, its overlap with the study-specific signatures ranged from 3-40%. The excluded genes are likely to be cohort-specific findings that can not be replicated. Second, the meta-signature recruited 41 genes not previously picked by any of the single cohort signature, likely representing predictive features with small but consistent effects previously masked in single studies. When examining the performances of the gene signatures, the meta-signature showed a comparable or better performance compared with the individually optimized signatures both in the odds ratio estimates (Bottom row of Table 3) and in Kaplan-Meier analysis (FIG. 3). This shows that the meta-signature can serve as a common breast cancer recurrence index that is able to predict patient survival in heterogeneous sample populations. When a gene signature built in one study cohort performs differently in another, such meta analysis provides a solution to identify a cross-study validated expression signature that holds across independent samples.

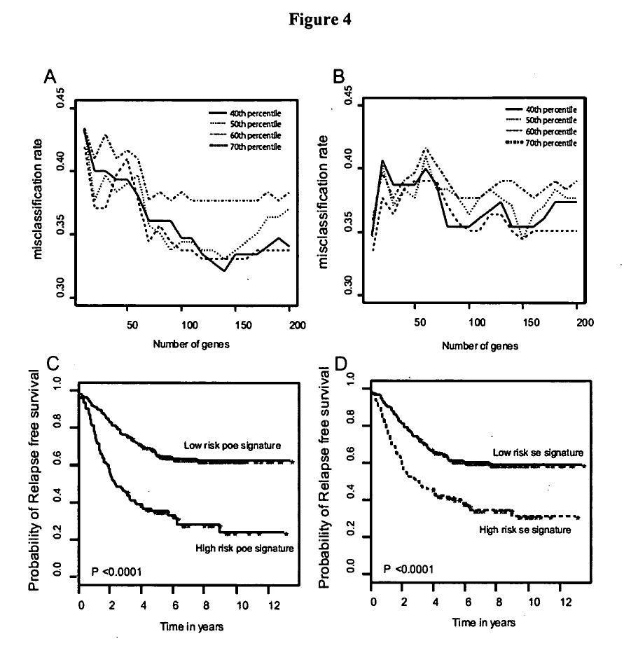

Comparison of data integration based on poe transformation and simple linear resealing. An alternative approach to integrating data across multiple datasets is to perform a study-wise global normalization. For one study, let be the globally scaled expression value for gene j in sample i. Each study dataset is then standardized to have zero mean and unit standard deviation. The linearly rescaled values can also be used for data integration purposes in that expression values generated from different array platforms are standardized to a common scale. Such an approach is less computationally challenging compared to the mixture model-based rescaling described in the previous sections. However, there are several advantages to the mixture model-based transformation. First, the method incorporates biological information into estimating the posterior probabilities of expression. The transformed values carry meaningful interpretations as signed probabilities of differential expression of a gene in a particular sample. Second, the underlying normal and uniform mixture distributions give equal density in the tails and is effective in reducing the influence of extreme expression values. And third, the Bayesian hierarchical modeling approach borrows strength across genes resulting in shrinkage-type estimators for a large correlated gene-specific parameter vector. This is a method in which the high dimensional gene expression data are de-noised. To study the benefit of data integration based on poe compared to that based on the linearly rescaled values, the model performances were compared based on data integration by these two methods. FIG. 4A shows that with the poe transformation, misclassification rates steadily decreases as more genes are used in the classifier. Performance based on linearly rescaled data (FIG. 4B), however, is unpredictable. FIGS. 4C and 4D uses a 90-gene metasignature based on poe and based on the global standardization respectively in predicting survival. The signature based on poe is noticeably better than the signature based on global standardization in differentiating patients at low risk of recurrence from those at high risk of recurrence. Taken together, the poe transformation outperforms the linear rescaling method in combining multiple microarray data sets. The meta-signature identified based on poe values therefore offers more reliable prediction of recurrence-free survival in the meta-cohort.

The meta-signature displays two distinct expression patterns. A heat map representation of the poe profile for the 90 gene meta-signature revealed two distinct patterns of differential expression. Genes in the top half of the matrix displayed consistently high probability of overexpression (yellow) in the recurrent samples (R). On the other hand, genes in the bottom half displayed great probability of under-expression (blue) in the recurrent group. Individually generated heat maps of the raw data confirmed such distinct patterns at raw measurement levels. Functional annotation revealed genes involved in many important biological processes such as cell cycle regulation (e.g., CDC28 protein kinase regulator subunit 2), cell adhesion (e.g., chemokine C-X3-C motif receptor 1), and apoptosis (e.g., secreted frizzled-related protein 4). Some of the genes in the meta-signature were previously shown to correlated with breast cancer survival outcome. For example, Keyomarsi et al. (Keyomarsi et al., N Engl J Med 2002, 347:1566-75) demonstrated the association of the cell cycle regulator cyclin E and death due to breast cancer.

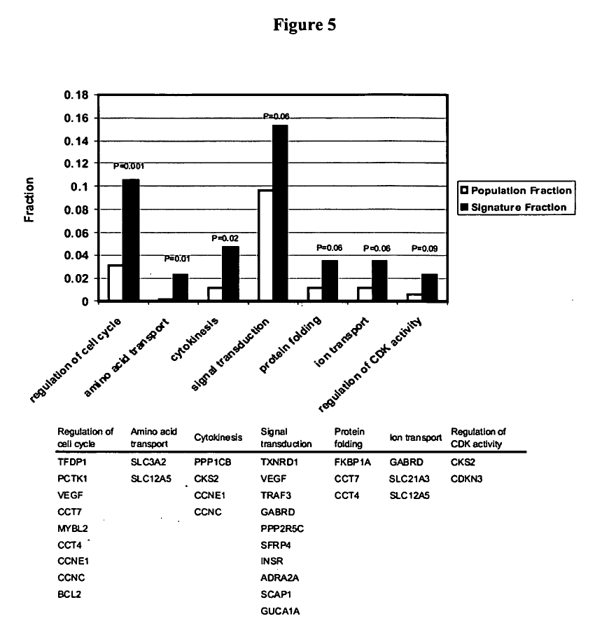

Enriched functional classes in the meta-signature. To gain a better understanding of the processes related to disease recurrence, it was examined whether a particular functionally defined biological process is enriched in the recurrence signature. Each of the ninety genes were mapped to Gene ontology (GO) terms and then grouped by functional classes. Based on the hypergeometric distribution, we calculated the significance of over-representation of a particular process in the signature. FIG. 5 demonstrates the top seven enriched functional groups in the meta-signature, comparing the total proportion (out of 2310 annotated) and the signature proportion (out of 85 annotated) of genes in each group. Cell cycle regulation is the most highly over-represented category (P=0.001). All genes under this category except BCL2 displayed increased expression level, reflecting elevated cell cycle activities. Signal transduction represents the largest functional class over-represented in the meta-signature. Genes involved in signalling pathways that regulate cell growth (VEGF, PPP2R5C), immune response (TRAF3), apoptosis (SFRP4), and other processes are found to constitute 15.7% of the meta-signature compared to the 9.7% in the entire gene set (the common set).

TABLE 1
Breast cancer gene expression data sets used in the prognostic meta-analysis.
Bad outcome (Y = 1) is defined as recurrence during follow-up, and good outcome
(Y = 0) is defined as remaining recurrence-free for at least three years.

| Authors | Array platform | Number of array elements | Sample size (n) | Good outcome (n₀) | Bad outcome (n₁) |
|---|---|---|---|---|---|
| Sorlie et al. | Spotted cDNA | 8102 | 58 | 23 | 35 |
| van't Veer et al. | Inkjet oligonucleotide | 25000 | 78 | 44 | 34 |
| Sotiriou et al. | Spotted cDNA | 7650 | 98 | 53 | 45 |
| Huang et al. | Affymetrix chip | 12625 | 71 | 36 | 35 |

TABLE 2
Comparisons of the number of genes (Size), the number of elements
overlap with the meta-signature (overlap), and the prediction error rates
for the signatures identified in individual study cohort and in the
meta-cohort.

| | Sorlie | van't Veer | Sotiriou | Huang | Meta-cohort |
|---|---|---|---|---|---|
| Size | 10 | 60 | 90 | 140 | 90 |
| Overlap | 4 | 14 | 19 | 6 | — |
| Prediction error rate | 0.28 | 0.29 | 0.35 | 0.18 | 0.33 |

TABLE 3
Comparison of the performances of the individual signatures and the meta-signature in each single study
cohort. Table lists odds ratios (95% confidence interval) comparing the odds of actual recurrence for
those being classified as high risk to the odds of recurrence for those being classified as low risk of
recurrence by each signature.

| | Cohort | | | | | | | |
|---|---|---|---|---|---|---|---|---|
| | Sorlie | | van't Veer | | Sotiriou | | Huang | |
| Signature | (n = 58) | | (n = 78) | | (n = 98) | | (n = 71) | |
| Sorlie (D = 10) | 18.6 | (5.0, 69.5) | 2.1 | (0.8, 5.4) | 2.3 | (1.0, 5.3) | 10.87 | 3.5, 33.8) |
| van't Veer (D = 60) | 3.1 | (1.1, 9.2) | 10.6 | (3.3, 33.9) | 4.1 | (1.7, 9.7) | 1.3 | (0.5, 3.4) |
| Sotiriou (D = 100) | 1.7 | (0.6, 5.0) | 3.5 | (1.4, 8.9) | 7.8 | (3.0, 20.1) | 1.5 | (0.6, 3.7) |
| Huang (D = 130) | 5.1 | (1.6, 15.7) | 2.3 | (0.9, 5.6) | 0.9 | (0.4, 2.0) | 184.9 | (30.1, 1137.2) |
| Meta (D = 90) | 25.0 | (4.2, 149.0) | 4.1 | (1.6, 10.6) | 6.0 | (2.5, 14.5) | 5.8 | (2.1, 16.5) |

D is the number of genes in a signature. n is the sample size for each cohort.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method, comprising:
a) providing a plurality of microarray data sets, wherein each data set represents microarray profiling of a distinct sample, and wherein said sample is representative of a disease state;
b) performing a two stage Bayesian mixture modeling calculation on said data sets to generate probability of expression (poe) matrices for each data set;
c) combining said poe matrices to generate a combined data matrix; and
d) generating a prognostic meta signature for said disease state based on said combined data matrix.

2. The method of claim 1, wherein said disease is cancer.

3. The method of claim 2, wherein said cancer is breast cancer.

4. The method of claim 2, wherein said prognostic meta signature is indicative of increased probability of relapse free survival of cancer.

5. The method of claim 2, wherein said prognostic meta signature is indicative of decreased probability of relapse free survival of cancer.

6. The method of claim 2, wherein said prognostic signature is indicative of cancer likely to metastasize.

7. The method of claim 1, wherein said microarray data sets comprise gene expression data.

8. The method of claim 1, wherein said prognostic signature comprises expression data for at least 20 genes.

9. The method of claim 1, wherein said prognostic signature comprises expression data for at least 50 genes.

10. The method of claim 1, wherein said prognostic signature comprises expression data for at least 100 genes.

11. The method of claim 1, wherein said prognostic signature comprises expression data for at least 500 genes.

12. A prognostic meta signature comprising normalized gene expression data from at least two independent gene expression profiling studies, wherein said gene expression data comprises data from microarray profiling of a distinct sample, and wherein said sample is representative of a disease state.

13. The prognostic meta signature of claim 12, wherein said prognostic signature is indicative of increased probability of relapse free survival of cancer.

14. The prognostic meta signature of claim 12, wherein said prognostic signature is indicative of decreased probability of relapse free survival of cancer.

15. The prognostic meta signature of claim 12, wherein said prognostic meta signature comprises expression data from at least 3 independent gene expression profiling studies.

16. The prognostic meta signature of claim 12, wherein said prognostic meta signature combines probability of expression matrices from said at least 2 independent gene expression profiling studies.

17. The prognostic meta signature of claim 16, wherein said probability of expression matrices are generated using two stage Bayesian mixture modeling calculation on normalized data sets from said at least 2 independent gene expression profiling studies.

18. The prognostic meta signature of claim 12, wherein said disease is cancer.

19. The prognostic meta signature of claim 18, wherein said cancer is breast cancer.

20. The prognostic meta signature of claim 12, wherein said prognostic signature comprises expression data for at least 20 genes.

21. The prognostic meta signature of claim 12, wherein said prognostic signature comprises expression data for at least 50 genes.

22. The prognostic meta signature of claim 12, wherein said prognostic signature comprises expression data for at least 100 genes.

23. The prognostic meta signature of claim 12, wherein said prognostic signature comprises expression data for at least 500 genes.

24. A method of screening compounds, comprising:
a) providing
i) a cell; and
ii) one or more test compounds; and
b) contacting said cell with said test compound;
c) generating a gene expression profile of said cell in the presence and absence of said test compound; and
d) comparing said gene expression profile to a prognostic meta signature generated by the method of claim 1.

25. The method of claim 24, wherein said cell is a cancer cell.

26. The method of claim 24, wherein said prognostic meta signature represents gene expression profiles of cancer cells.

27. The method of claim 24, wherein said cell is in an animal.

28. The method of claim 27, wherein said animal is a non-human mammal.

29. The method of claim 28, wherein said animal is a human.
